(19) United States
(12) Patent Application Publication
(10) Pub. No.: US 2006/0075648 A1
(43) Pub. Date: Apr. 13, 2006

(54) Device for measuring a human head

(76) Inventors: Joseph R. Madsen, Newton, MA (US); James H. Goldie, Lexington, MA (US); Kristen Choppy, Somerville, MA (US); Thieu Q. Truong, North Easton, MA (US)

Correspondence Address: SCHULTE ROTH & ZABEL LLP;ATTN: JOEL E. LUTZKER, 919 THIRD AVENUE, NEW YORK, NY, 10022, US

(21) Appl. No.: 11/234,355

(22) Filed: Sep. 23, 2005

Related U.S. Application Data
(60) Provisional application No. 60/612,414, filed on Sep. 23, 2004.

(51) Int. Cl. G01B 3/34; G01B 5/02; A61B 5/107

(52) U.S. Cl. 33/512; 600/587

(57) ABSTRACT

The invention relates to apparatus and methods for measuring a human head. The apparatus disclosed herein involve the use of linear rulers, or rotating contacting members, coupled to a support member. The apparatus disclosed herein can be positioned on a human head, using the methods disclosed herein, to determine the outer contour of the head. Such a measurement can aid in the diagnosis and monitoring of various abnormalities of the human head, including plagiocephaly.

### CROSS-REFERENCE TO RELATED APPLICATIONS

This application claims the benefit of United States Provisional Application No. 60/612,414, filed Sep. 23, 2004.

### BACKGROUND OF THE INVENTION

A need exists for a cost efficient method to measure and follow the shape of the heads of infants. Measuring head or cranial shape has been a mainstay in the treatment of cranial synostosis for decades, but with the increasing prevalence of positional plagiocephaly it has become a major reason for referral to neurosurgical clinics by pediatricians and by parents. Conditions of the head such as sagittal synostosis resulting in a long and narrow head, can be suspected or even diagnosed on the basis of cranial measurements, as can the situations of flattening of the back or asymmetrical flattening of the head related to the position in which a baby sleeps. Orthotic devices are often used to treat the conditions which do not actually require surgery, and the ability to make inexpensive and repeated measurements would be useful in evaluating the need for treatment of a particular patient as well as the response to treatment of any individual patient, and the effectiveness of any particular therapeutic intervention.

Traditionally, measurements of cranial shape have been made either on radiographs which require radiation exposure, or with the use of large calipers which require the points of the calipers to be placed at particular locations on an infant's head. This introduces significant operator dependency on the accuracy of the measurement, and purchase of the calipers has limited their use generally to highly skilled neurosurgeons and others who see a large number of these cases.

Furthermore, diagnosis of cranial abnormalities and/or diseases are often made on physical exam by clinicians without quantifying the shape of the outer contour of the subject's head. This makes it difficult for the clinician to accurately diagnose a cranial disease and/or abnormality and makes it difficult for the clinician and/or parents to track the progression or regression of a particular subject's cranial disease and/or abnormality.

### SUMMARY OF THE INVENTION

One aspect of the invention is an apparatus for measuring an outer contour of a human head, comprising a support member and a plurality of contacting members for contacting a human head.

In a particular embodiment of the invention, at least one of the contacting members is pivotally mounted on the support member. In a further embodiment, the contacting members are adjustable. In another embodiment of the invention, the apparatus comprises at least one radially adjustable contacting member. In one embodiment of the invention, the apparatus comprises at least 1 adjustable contacting member. In another embodiment of the invention, at least one of the contacting members is rotatably mounted on the support member. In yet another embodiment the apparatus comprises at least one contacting member. In a further embodiment, the apparatus comprises at least 4 contacting members. In yet another embodiment, the apparatus comprises at least 12 contacting members. In one embodiment of the invention, the apparatus comprises a support member and a plurality of contacting members rotatably coupled to the support member wherein each of the plurality of contacting members comprises a curved surface for contacting with the human head and wherein the curved surface of each contact member has a radius of curvature that is one of increasing and decreasing in a direction of rotation. In a specific embodiment, the curved surface of the contacting member comprises a portion which is characterizable by a spiral equation. In a further embodiment, the spiral equation characterizes a linear spiral. In another embodiment the spiral equation characterizes a logarithmic spiral. In another embodiment of the invention, the apparatus comprises a support member and a plurality of adjustable contacting members coupled to the support member, wherein each of the plurality of adjustable contacting members comprises a surface for contacting with the human head, at least one of the adjustable contacting members comprises a mechanism for locking the adjustable contacting member. In another embodiment of the invention, the apparatus comprises a support member and a plurality of adjustable contacting members coupled to the support member, wherein each of the plurality of adjustable contacting members comprises a surface for contacting with the human head, the support member comprises a mechanism for locking at least one of the adjustable contacting members. In another embodiment, the apparatus comprises a locking mechanism. In a further embodiment, the support member and/or the contacting members comprise a locking mechanism.

In yet another embodiment of the invention, the apparatus comprises a support member and a plurality of radially adjustable contacting members coupled to the support member, wherein a contact surface of the radially adjustable contacting members to be contacted with the human head moves radially. In another embodiment of the invention, the apparatus comprises a support member and at least four adjustable contacting members coupled to the support member, wherein each of the at least four adjustable contacting members comprises a surface for contacting with the human head and the adjustable contacting members have indicia thereon for determining the outer contour of a human head. In a specific embodiment, the contacting members are radially adjustable.

In another embodiment of the invention, the apparatus comprises a support member and at least four adjustable contacting members coupled to the support member, wherein each of the at least four adjustable contacting members comprises a surface for contacting with the human head and the support member has indicia thereon for determining the outer contour of a human head. In a specific embodiment, the contacting members are radially adjustable.

The invention also encompasses methods of determining the outer contour of a human head and of detecting the presence or absence of cranial abnormality and/or disease.

One embodiment of the invention comprises positioning about the human head a plurality of contacting members rotatably coupled to a support member; adjusting each of the contacting members such that a curved surface of the contacting member having a radius of curvature that one of increases and decreases in a direction of rotation touches a surface of the human head; and/or measuring the contour of the human head after the plurality of contacting members have been adjusted to contact the human head.

Another embodiment of the invention comprises positioning about the human head a plurality of contacting members that are coupled to a support member; adjusting each of the adjustable contacting members such that a surface of the contacting member touches a surface of the human head; and/or measuring the contour of the human head using indicia on at least one of the adjustable contacting members after the plurality of adjustable contacting members have been adjusted to contact the human head.

A further embodiment of the invention comprises the steps of positioning about the human head a plurality of contacting members that are coupled to a support member; adjusting each of the adjustable contacting members such that a surface of the contacting member touches a surface of the human head; and/or measuring the contour of the human head using indicia on the support member after the plurality of adjustable contacting members have been adjusted to contact the human head.

Another embodiment of the invention comprises positioning about the human head a plurality of adjustable contacting members that are coupled to a support member; adjusting each of the adjustable contacting members such that a surface of the adjustable contacting member touches a surface of the human head; measuring the contour of the human head after the plurality of adjustable contacting members have been adjusted to contact the human head; and/or based on the measurements, detecting the cranial abnormality in the human head.

The invention also contemplates a system for measuring a head comprising an apparatus, wherein the apparatus comprises a support member and adjustable contacting members, and a readout tray in which the apparatus is placed. The readout tray has a plurality of sensors which correspond to the contacting members on the apparatus. In a particular embodiment, the readout tray comprises alignment pins which couple with holes in the apparatus.

### BRIEF DESCRIPTION OF THE DRAWINGS

FIG. 1. depicts a top-down view of a portion of the support member 1, a window in the top ring 2 , a portion of the sweatband 3, a linear ruler 4, an indicator 5 and a spring loaded set screw 6.

FIG. 2. depicts an underside view of a portion of the support member 7, a fastener 8, and a ramp 9 for tightening and locking the sliding rulers in place.

FIG. 3a. depicts an apparatus with a handle 10, and a plurality of radially adjustable wedge shaped contacting members 11, mounted to a support member 12, using a magnetic contacting surface 13.

FIG. 3b. depicts a radially adjustable contacting member 14, with indicia 15, and a pointer 16 that is part of a support member.

FIG. 3c. depicts a cross sectional view of an apparatus showing an indicator 17, a support member 18, a radially adjustable contacting member 19, and a magnetic contacting surface 20.

FIG. 4. depicts an apparatus for measuring a head 21 and a readout tray 22 with an array of sensors 23 and alignment pins 24.

FIG. 5. depicts a portion of an apparatus with a transparent plastic strip 25 for changing the reference diameter of the apparatus, a slot in the plastic strip 26 to allow position adjustment, an adjustable contacting member 27 with pivot 28, a tab on the plastic strip 29 to adjust the azimuthal position of the plastic strip, reference indicia 30 and a base ring 31.

### DETAILED DESCRIPTION OF THE INVENTION

The invention allows rapid collection of multiple measurements on a subject's head in a non-invasive way. The apparatus and methods disclosed herein can be easily used by primary care pediatricians and other clinicians, and also by parents who wish to follow suspected cranial abnormalities in their children or simply to assess the degree of flattening in positional plagiocephaly.

One aspect of the invention comprises an apparatus consisting of a support member that is capable of being positioned about a subject's head, the support member having mounted to it a plurality of contacting members that can come into contact with the subject's head. The apparatus is placed in the transverse plane above the subject's orbital sockets and ears. The contacting members may be adjustable so that they can be brought into and out of contact with the subject's head. Once the contacting member contacts the subject's head, a measurement can be made based on the position of the contacting member. From these measurements, a representation of the outer contour of the subject's head can be derived, which can then be used for diagnosis or sequential monitoring.

As more contacting members are mounted on the apparatus, more data points can be derived, and a more detailed representation of the outer contour of the subject's head can be made. The contacting members provide the clinician with data that can be used to calculate the shape of the outer contour of the subject's head. The data provided by the contacting members can be linear distance measurements provided in units such as millimeters, centimeters, decimeters, meters and inches, and/or angular measurements provided in such units as radians and degrees.

Another aspect of the invention provides methods of measuring the outer contour of a subject's head and diagnosing and/or detecting the presence of a cranial abnormality or disease. These methods, as well as how to interpret the data collected using the apparatus and methods of the invention, are described in more detail below.

Support Member

The support member is a location for mounting a plurality of contacting members. The invention contemplates a support member suitable for mounting about a human head. In certain embodiments of the invention where the apparatus comprises an elliptical support member or a support member that substantially conforms to the elliptical outer contour of a human head, the apparatus is oriented about the subject's head so that the support member substantially matches the elliptical contour of a human head when viewed in the horizontal or transverse plane. Thus, such support members will have anterior and posterior portions corresponding to the anterior and posterior portions of the subject's head and two lateral portions of the support member corresponding the lateral portions of the subject's head.

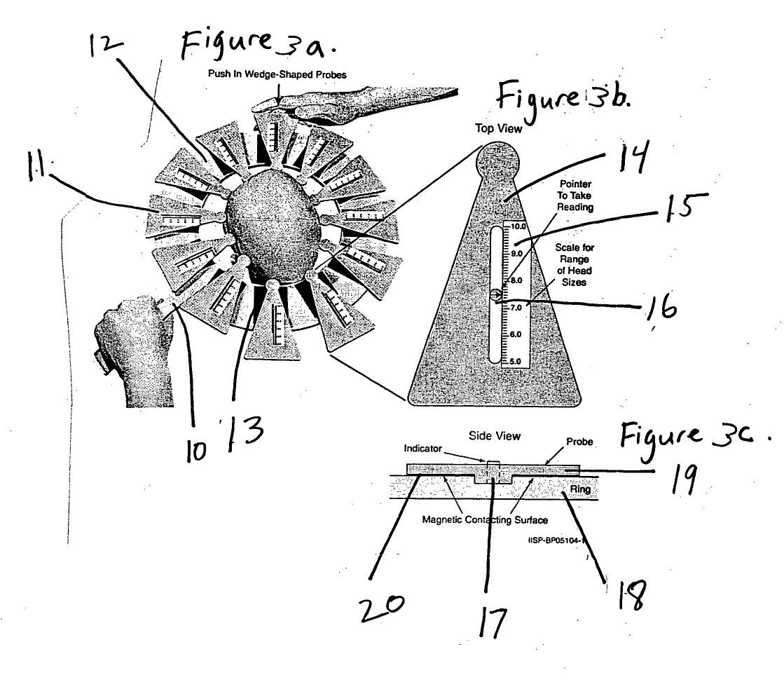

Optionally, a handle can be attached to the support member, see FIG. 3a., which allows the user to grasp and position the device with one hand while manipulating the contacting members with the other hand.

Contacting Members

One aspect of the invention comprises an apparatus comprising a support member having mounted to it a plurality of contacting members that can come into contact with the subject's head. The contacting members are either adjustable, so that they can be brought into and out of contact with the subject's head while keeping the support member stationary, or fixed. Once the contacting member contacts the subject's head, a measurement can be made based on the position of the contacting member. The distance measured can be between the subject's head and a point on the support member.

In one embodiment, the contacting members are linear rulers which can be adjusted to contact the subject's head. The contacting members can have indicia thereon for determining the distance between the support member and the subject's head. Alternatively, the support member can be marked with indicia in order to determine the distance between the support member and the subject's head. The rulers can be adjusted radially inwards and outwards to and from the central area of the plane defined by the support member. In another embodiment, the contacting members are shaped as spirals and are rotabaly mounted on the support member so that they rotate to come in contact with the subject's head, and when the rotation is such that the spiral contacting member touches the surface of the subject's head, the angle of rotation of the spiral contacting member can be used to determine the radius formed by the spiral contacting member and the subject's head.

As a spiral contacting member is rotated, the radius of curvature of the contacting member's contacting surface either increases or decreases in the direction of rotation. Thus, knowing the equation that represents the radius of curvature of the contacting surface of the spiral contacting member and the angle of rotation of the contacting member, one can determine the radius from the pivoting point of the spiral contacting member to the subject's head and the tangent line at the contact point of the spiral contacting member and the subject's head.

The angle of rotation of the contacting member can be determined using indicia either on the contacting member or the support member. When the indicia are present on the support member, the contacting member can have a window for viewing the angle of rotation measurement. Indicia can also be viewed using any of the display technologies disclosed herein below, or known to one skilled in the art.

The essential feature of each spiral contacting member is that different points along its contacting surface will represent a different radius and a different tangent line. Any spiral shape defined by any spiral equation is suitable for use in the invention. By way of example and not limitation, a linear spiral and a logarithmic spiral are suitable spiral shapes for use in the invention.

There is inherently a small unusable part of the spiral and it is also desirable to have rounded edges to minimize the risk of skin irritation from a sharp point. In one embodiment of the invention, the unusable part of the spiral is a smooth polynomial with smooth first derivative.

The spiral contacting members can be mounted to pivot in the same plane as that created by the support member, or can alternatively be mounted to rotate perpendicular to the plane created by the support member.

By arraying a plurality of contacting members around a support member, a numerical representation of the outer contour of the head can be made, which can be used to create a graphical representation. The more spiral contacting members that are arrayed around the support member, the more detailed a representation of the outer contour of the skull can be achieved. Either the numerical or graphical representation of the outer contour of the head can be used for diagnosis and sequential follow-up purposes.

Locking Mechanisms

In order to facilitate the gathering of data provided by the invention, in one embodiment of the invention, the apparatus comprises a locking mechanism. In another embodiment, the apparatus comprises a plurality of locking mechanisms.

The locking mechanism can be used to lock a contacting member in place once the apparatus has been positioned on the human head. Such a locking mechanism facilitates removal of the apparatus without inadvertently moving the contacting members. Once removed, data from the contacting members can be analyzed. In one embodiment of the invention, a locking mechanism locks all of the contacting members. In another embodiment, each contacting member locks independently.

Any locking mechanism known to one skilled in the art can be used in the invention, including but not limited to a magnet, an electromagnet, a ratchet, a clamp, a screw, a pin, a bolt and a spring. The locking mechanism can also comprise a release mechanism whereby the locking mechanism is disengaged, allowing the contacting member to move again.

The locking mechanism can also, at least in part, be separate from the apparatus. For example, a clamp that is separate from the apparatus could be attached to the support member to cover the contacting members, thereby locking the contacting members in place.

Readout

A method of reporting the position of spiral contacting members most simply could be printed numbers and/or indicia around the perimeter with a pointer indicating the number amount of rotation of the contacting member. The pointer can be attached to the pivot, or it can be part of the pivot, so that it remains stationary while the spiral contacting member is rotated. Alternatively a window could be made in the spiral so that the number would be read on the ring. Alternatively, the pointer and/or numbers and/or indicia also be placed on the support member. For each linear ruler contacting member, the pointer and/or numbers and/or indicia can be placed either on the support member, the contacting members, or both. A window can also be used, either on the support member or on the contacting member, to read the number.

Materials

The apparatus comprising the support member and contacting members can be constructed of any suitable material, by way of example and not limitation, foam, metal, metal alloys, plastic, paper, rubber or cardboard, or any combination thereof and can be translucent or opaque.

Utilization

The invention provides methods of determining the outer contour of a subject's head. In one embodiment of the invention, the apparatus is positioned in the transverse or horizontal plane of the subject's head. In another embodiment of the invention, the apparatus positioned in the plane of the largest anterior-posterior (AP) and lateral diameters of the subject's head, which allows for standardization of measurements between patients.

The following example of a method for determining the outer contour of a subject's head is given by was of example and not limitation:
The apparatus is placed to be in the plane of the subject's head above the orbital rims and above the ears. The contacting members in the AP dimension are set first. The AP contacting members can be comprised of as few as a single pair of contacting members (one contacting member in the anterior portion of the apparatus and one contacting member in the posterior portion of the apparatus). In order to center the head in the AP direction, the AP contacting members are either individually manually turned so that they are equivalently adjusted (i.e., they are identically spaced from the support member to the head) or the contacting members are connected such that a single adjustment ensures that they are equivalently adjusted. The AP contacting members are adjusted so that they just come into contact with the subject's head. Next, the lateral contacting members are similarly adjusted to center the head in the lateral direction. Each of the other contacting members are then adjusted to just contact the contacting member with the subject's head. Once all of the contacting members arrayed on the support member are adjusted to contact the subject's head, the contacting members can optionally be locked in place. At this point, the entire ring is removed from the subject's head and the results are recorded. The results can then be graphed to create representation of the outer contour of a subject's head.
   Interpreting the Data

The apparatus disclosed herein provides data from each contacting member. If the contacting member is a linear ruler, the data provided will be linear distance from a reference point on the support member to the subject's head. If the contacting member is a spiral contacting member, these data can be a linear distance and/or an angular measurement such as degrees or radians.

For example, once contacted with the human head, spiral contacting members can provide an angular measurement. Once the angular measurement is determined, the radius from the pivot point of the contacting member to the head and the tangent line at the contact point can calculated using the equation representing the curved contacting surface of the spiral contacting member.

Once the dimensions of the subject's head are determined with the device, the results can be graphed on appropriately ruled graph paper, for example, customized polar coordinate form or Cartesian form. Alternatively, the data provided by the invention can be entered into a calculator or computer that can graph and/or interpret the data. Once the linear distance between each contacting member and the subject's head is known, a clinician or parent for example can interpret the data and detect the presence or absence of a cranial disease or abnormality.

### RADIAL SLIDING RULER EMBODIMENT

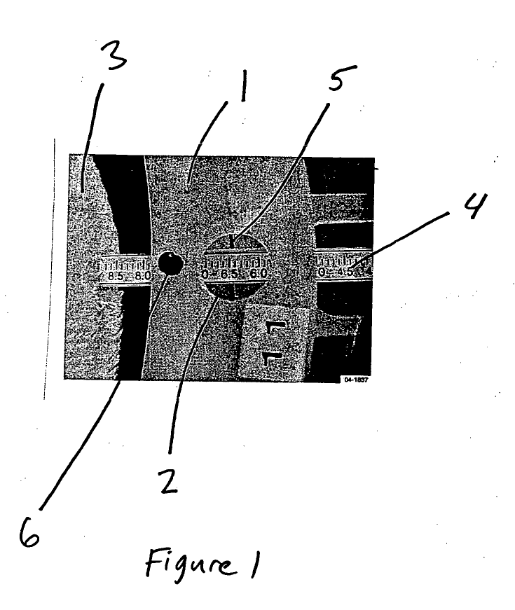

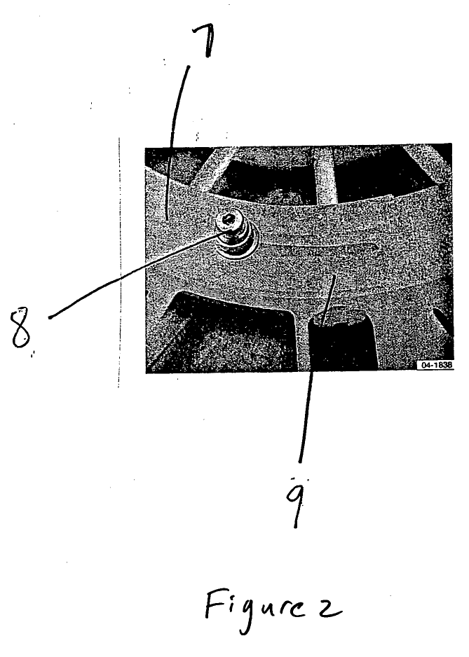

In a particular embodiment of the invention, the support member of the device is comprised of three rings. The middle ring has a plurality of radial troughs, which provide guidance for the rulers as they slide in and out radially. The top ring of the device both retains the rulers in their troughs and clamps down on the rulers when it is necessary to lock their positions. In addition, as shown in FIG. 1, there is a small window in the top ring for each ruler that allows the user to view the position of the ruler and record the measurement manually. On the underside of the bottom ring, as shown in FIG. 2, there is a plurality of ramps or inclines (e.g., 6). As the bottom ring is rotated relative to the top ring, the ramps tighten on the heads of a plurality of fasteners (e.g., 12), putting them in tension. Since the fasteners engage the threads in the top ring, the tension pulls the top ring against the top side of the rulers, causing their positions to be locked.

The inner ends of the rulers that contact the head are, for example, spherical in shape in order to minimize discomfort of the subject. The inner ends of the rulers that contact the head are connected to each other by an elastic band, or a plurality of elastic bands. The band is, for example, an elastic sweatband. The attached elastic band tends to force the sliding rulers simultaneously radially inward. A washer is optionally attached to the inner ends of the rulers so that the spheres at the end of the rulers do not pull through the elastic band.

Application of the device to the subject's head is accomplished either 1) by opening the sweat band by hand and then gently placing the device around the subject's head or 2) by moving and locking the rulers in their outermost position, placing the device over the subject's head, releasing the lock, and then allowing the rulers to move into contact with the subject's head. Once the spheres of the rulers contact the head, the rulers are locked again by the user and the device is removed from the head.

The rulers are marked such that the reading indicated in the windows of the device represents the distance from the center of the ring to the inside surface if the spherical contact surface of the ruler.

In order to assist the top ring of the device to uniformly clamp all rulers simultaneously, ball plungers, or set screws with spring loaded (plastic) tips, are mounted in threaded holes in the top ring, with the tip of the ball plunger bearing against the top surface of each ruler. The position of the ball plungers is adjusted until the ball plunger exerts enough force to lock the position of the ruler when the lock mechanism of the device is in effect. The ball plungers thereby prevent the rulers from shifting positions once the device is locked and is being removed from the head.

In a particular embodiment, the device is comprised of 12 radially adjustable linear rulers arrayed at 30 degree intervals in azimuth. The range of measurement for each ruler is, for example, between 10 to 20 cm or between 10 to 15 cm.

### RADIAL SLIDING RULER EMBODIMENT UTILIZING MAGNETS

In another embodiment of the invention, the device comprises a support member and a plurality of radially adjustable contacting members wherein magnets are installed at the contact interface between the contacting members and the support member. The magnets in this embodiment provide the necessary friction to preserve the positions of the contacting members once they have been brought in contact with the head.

In one embodiment, one or more rivet-like fasteners or guide pins, installed in the support member and which protrude into radial grooves in each of the contacting members, serve to guide the motion of the contacting members as they are moved radially inwards and outwards.

In another embodiment, radial guidance can be achieved by a tongue and groove arrangement at the contact interface between each contacting members and the support member. In a specific embodiment, a small square ridge on the underside of the contacting members slides inside a radial trough in the support member.

Head Measurement System Using Readout Tray

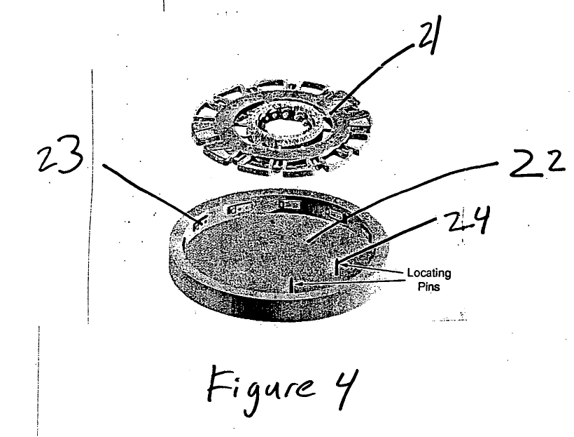

One embodiment of the invention comprises a head measurement system comprising a linear ruler device as described herein above and a readout tray wherein the device which has been adjusted to measure a subject's head, locked and then removed from the subject's head, is lowered into a readout tray. An array of sensors, for example infrared sensors, is placed around the periphery of the tray. These sensors provide a measure of the distance between the outer ends of the rulers and the sensors installed in the tray. In a specific embodiment, the number of sensors equals the number of contacting members. The analog measurements are then wired into a digital to analog converter which has a serial output to a computer or PDA. In one embodiment, Sharp model GP2D120 infrared sensors are utilized in the tray. In another embodiment, Texas Instruments TLC545 19-input analog to digital 8-bit converter is utilized. In a specific embodiment, the readout tray comprises sensors, an analog to digital converter and/or a computer. See FIG. 4.

The readout tray also comprises a plurality of alignment pins which fit into receptacles on the support member of the device in order to insure correct placement of the device into the readout tray. Alternatively, the support member comprises a plurality of alignment pins which fit into receptacles on the readout tray.

Adjustable Reference Diameter

In one embodiment of the invention in which the contacting members are rotatably coupled to the support member, the head is measured by measuring the departure of the head measurements from a nominal circle at each contacting member location. The resulting values are clearer to the user since measurements such as “+7 mm” or “−3 mm” would be obtained, rather than measurements like “67 mm.”

The choice of the circle as the nominal shape is reasonable, since the cephalic index (ratio of ML to A-P) varies significantly and still be considered “normal,” depending on a number of factors, including ethnicity. The invention contemplates that other shapes can be used as a nominal shape.

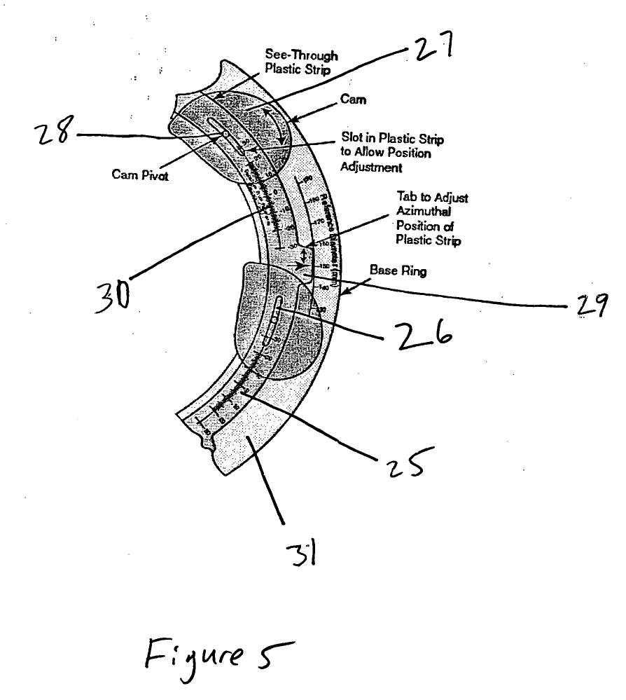

Given the range in head sizes, with multiple reference diameters, a closer fitting circle can then be chosen for each subject, resulting in a set of measured departures with smaller absolute values. Multiple reference diameters can be accomplished in a number of ways according to the invention. The most obvious is to have apparatus of different sizes each with its unique reference diameter (e.g., 12 mm, 15 mm, 18 mm) and sufficient contacting member range to overlap slightly with the next-sized apparatus. Alternatively, as shown in FIG. 5, the invention contemplates incorporation of multiple reference diameters into a single device:

In this embodiment, the device includes a clear, see-through plastic ring placed over the contacting members. Identical scales (“Departure from reference diameter (mm)”) for each contacting member are marked on the plastic ring at regular intervals along the circle that passes through the pivot point of each contacting member. The midpoints of these scales are marked “0.” A single scale (labeled, e.g., “Reference diameter (mm)”) is marked on the top surface of the base ring of the apparatus.

In order to adjust the reference diameter, the user simply adjusts the azimuthal position of the see-through ring. A pointer on the edge of the moveable plastic ring indicates the reference diameter on the “Reference Diameter (mm)” scale. The numerical value at the intersection of the edge of the contacting member with the “Departure from reference diameter (mm)” scale provides a measure of the departure of the contacting member position from the reference diameter. In this embodiment, the user would record a reference diameter and the departure value for each contacting member.

We claim:

1. An apparatus for determining an outer contour of a human head, comprising a support member and at least three radially adjustable contacting members coupled to the support member, wherein the at least three radially adjustable contacting members are connected to each other by an elastic band.

2. The apparatus of claim 1, wherein the support member comprises a mechanism for locking the contacting members in place.

3. An apparatus for determining an outer contour of a human head, comprising a support member and twelve radially adjustable contacting members coupled to the support member, wherein the radially adjustable contacting members are connected to each other by an elastic band.

4. The apparatus of claim 3, wherein the support member comprises a mechanism for locking the contacting members in place.

5. The apparatus of claim 3, wherein the radially adjustable contacting members are arrayed at 30 degree intervals around the support member.

6. The apparatus of claim 3, wherein the support member comprises a top ring, a middle ring and a bottom ring.

7. The apparatus of claim 6, wherein the top ring comprises windows for viewing the radially adjustable contacting members.

8. The apparatus of claim 6, wherein the middle ring comprises troughs in which the radially adjustable contacting members slide.

9. The apparatus of claim 6, wherein the bottom ring is fastened to the top ring by a plurality of fasteners.

10. A system for determining an outer contour of a human head, comprising a support member, at least three radially adjustable contacting members coupled to the support member, and a readout tray, wherein the readout tray comprises at least one sensor.
